(19) United States
(12) Patent Application Publication
(10) Pub. No.: US 2007/0280905 A1
(43) Pub. Date: Dec. 6, 2007

(54) Prognosis and Systemic Therapy for Treating Malignancy

(76) Inventors: Shulin Li, Baton Rouge, LA (US); Shiguo Zhu, Baton Rouge, LA (US); Denada Dibra, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 11/755,414

(22) Filed: May 30, 2007

Related U.S. Application Data
(60) Provisional application No. 60/809,876, filed on Jun. 1, 2006.

(51) Int. Cl. A61K 48/00; A61K 38/20; C12Q 1/68; G01N 33/574

(52) U.S. Cl. 424/85.2; 514/44; 435/6; 435/7.23

(57) ABSTRACT

It has been discovered that a sequential administration by intramuscular electroporation of the gene for IL12 followed about 10 days later by the gene for IL27 eradicated tumors in 33% of mice with aggressive 4T1 breast malignancy. In addition, the mice in which the initial tumor was eradicated rejected subsequent challenges with tumor cells. This simple sequential cytokine gene therapy can be used for treating residual malignancy and for inhibiting metastatic tumors. Tumors that respond to this treatment were shown to have the WSX-1 subunit of the IL27 receptor. The responsiveness of tumors containing WSX-1 to the dual IL12→IL27 therapy suggests that WSX-1 receptor may be used as a marker for selection of sensitive populations of tumor cells. Stat1 phosphorylation was shown to be a useful measure for WSX-1 activation in a panel of various cancer cells.

The development of this invention was partially funded by the Government under Grant No. CA098928 from the National Institutes of Health. The Government may have certain rights in this invention.

This invention involves the sequential delivery of gene therapy first with an initial administration of Interleukin-12 (IL12) gene followed days later by an administration of Interleukin-27 (IL27) gene for treating malignancy and the use of the molecular receptor WSX-1 to identify tumors responsive to this sequential therapy.

The development of tumor metastases is often associated with primary tumors and can be lethal. Many methods have been proposed to decrease or to inhibit the development and spread of systemic metastases. The conventional tumor therapy usually involves surgery or radiation to remove the primary tumor, which can leave microscopic tumor cells which then metastasize. To address the problem of metastasis, systemic chemotherapy and hormone depletion therapy are used following removal of the primary tumor. However, metastatic cells often develop a resistance to the chemical or hormonal therapy.

An alternative approach to decrease the development of metastasis is to increase the efficiency of the patient's own immune system. One method to induce a systemic immunity is the delivery to the primary tumor or otherwise to the patient of immunostimulating proteins or genes for those proteins. The resultant increase in immune response can both reduce the primary tumor mass and inhibit the development of a metastatic tumor. A simple and safe way to deliver a gene for an immunostimulatory protein to a patient is using plasmid DNA and electroporation which does not rely on a viral vector. The naked DNA-based, non-viral gene administration has the advantage of lower toxicity than delivery of high levels of the actual protein. A gene properly delivered will cause a persistent expression of the immunostimulatory protein, resulting in a persistent low level concentration of the protein. Delivery of the protein itself is not feasible because it would require an exceptionally high concentration delivered within a small amount of time due to the short half-life of the protein in the plasma, e.g., of the IL12 protein.

Among various systemic gene delivery approaches, injection of a gene into the skeletal muscle is preferable because a large bulk mass of muscle tissue is suited to uptake of expression vector and for the subsequent production and circulation of gene product into the blood for treating systemic malignancy (1). Moreover, skeletal muscle provides an accessible target for direct and effective gene delivery approach by electroporation (2). Injection of DNA into muscle followed by electroporation can increase the level of gene expression and thus protein formation by >100 fold (2).

An optimal electroporation delivery system was developed for intramuscular injection of a non-viral muscle expression vector containing a DNA nuclear localization signal and a potent muscle-specific promoter that generates high levels of gene expression. Using this delivery and expression system for increasing the cytokine, IL12, tumor growth was significantly inhibited regardless of the tumor models tested, but the tumors were not eradicated using an intramuscular injection (3, 4).

Interleukin-12

IL12 is produced by antigen-presenting cells, and the functional molecule of IL12 is a 70-kDa heterodimer protein composed of 40- and 30-kDa subunits. IL12 has multiple effects on immune cells. The major functions of IL12 are stimulation of IFN-γ production by natural killer (NK) and T cells, which in turn stimulates IL12 production and induction of anti-angiogenic genes. Its other important role is to exhibit immunoregulatory functions in the generation of T helper 1 (Th1) and cytotoxic T lymphocytes (CTL). For these reasons, systemic daily administration of IL12 recombinant protein generates a significant inhibitory effect on the metastatic growth of B16F10 melanoma and on established murine renal carcinoma (RENCA) and CT26 tumors (5). However, systemic delivery of IL12 protein is associated with severe toxicity in several experimental animal studies and in initial early-stage human trials, primarily because such a large dose of IL12 protein has to be used due to the short protein half-life. IL12 gene therapy has demonstrated greater efficacy and less toxicity than recombinant IL12 protein treatment in the RENCA tumor model (6). In addition, increased IL12 expression inhibits the expression of the tumor growth-promoting cytokine and growth factors, e.g., TGF-β and IL-10 (7). Therefore, IL12 gene therapy has a great potential for inhibiting metastatic tumor growth and for treating microscopic malignancy.

Systemic gene therapy using delivery of IL12 alone by intramuscular electroporation inhibited distal tumor growth but failed to eradicate established tumors (3). In contrast, intratumoral delivery of the IL12 gene via electroporation eradicated the established tumors (8). The difference in tumor eradication between the two delivery systems was shown not to be due to the difference in IL12 gene expression but was due to differences in the resulting cytokines at the tumor site, which determines the selection of immunity for eradicating tumors (4). Local tumor treatment with IL12 gene induced a dominant tumor-specific T cell immunity, while intramuscular IL12 gene therapy primarily induced NK cell-based anti-tumor activity (4). Therefore, one important element of eradicating tumors by systemic (intramuscular) IL12 gene therapy may be to enable the activation of tumor-specific T cells.

Interleukin-27

1327 is a novel member of the IL12 cytokine family. Similar to IL12, IL27 is a heterodimeric cytokine composed of p28 and EBI3 (Epstein-barr virus-induced gene 3) genes. These two proteins, EBI3 and p28, share high similarity with the p35 and p40 subunits of IL12, respectively. See U.S. Patent Application Publication No: US 2004/0219096, and International Application Publication No: WO 2005/079848. At the cellular level, IL27 has been shown to activate Stat1, Stat2, Stat3, Stat4 and Stat5 in naïve CD4 T cells. This activation has been associated with IL27 interaction with the cognate heterodimer receptor composed of WSX-1 and Glycoprotein 130 (19). Activation of Stat1 leads to the induction of T-bet and IL12Rβ2 by IL27 in naïve T cells. This induction is crucial for Th1 polarization. IL27 augments CD8 T effecter cells secreting granzyme B and induces Stat4-independent antitumor effect (9, 10).

We have discovered that a sequential administration by intramuscular electroporation of the IL12 gene followed about 10 days later by the IL27 gene eradicated tumors in 33% of mice with aggressive 4T1 breast malignancy. The tumor was eradicated by administering each gene only once. In addition, 83% of the mice in which the initial tumor was eradicated rejected subsequent challenges with tumor cells. This result using the aggressive 4T1 tumors is especially important because these tumors are considered to equal a grade IV malignancy in human patients. This simple sequential cytokine gene therapy can be used for treating residual malignancy and for inhibiting metastatic tumors. Tumors that respond to this treatment were shown to have the WSX-1 subunit of the IL27 receptor. Thus the presence of the WSX-1 subunit can be used to identify which tumors will respond to the sequential therapy of IL12 followed by IL-27.

### BRIEF DESCRIPTION OF DRAWINGS

FIG. 1 illustrates a photograph of groups of three mice with 4T1 tumors 18 days following administration via intramuscular electroporation to each group of either control plasmid (pCtr), plasmid with IL12 gene (IL12) or plasmid with IL27 gene (IL27), followed in ten days by administration with a second plasmid, as indicated.

FIG. 2A illustrates the effect of the various sequential gene therapies using IL12 and IL27 on 4T1 tumor volume for the groups of mice shown in FIG. 1.

FIG. 2B illustrates a Kaplan-Meier survival curve for the mice shown in FIG. 1.

FIG. 3A illustrates the change in tumor volume (mm³) in response to sequential gene therapy in CT26 mouse colon cancer tumors electroporated with IL12 followed in ten days by electroporation with IL12.

FIG. 3B illustrates the change in tumor volume (mm³) in response to sequential gene therapy in CT26 mouse colon cancer tumors electroporated sequentially with IL12 followed in ten days by IL27.

FIG. 3C illustrates the change in tumor volume (mm³) in response to sequential gene therapy in CT26 mouse colon cancer tumors electroporated sequentially with IL12 followed in ten days by IL27.

FIG. 3D illustrates the change in tumor volume (mm³) in response to sequential gene therapy in CT26 mouse colon cancer tumors electroporated sequentially with IL12 followed in ten days by IL27.

FIG. 3E illustrates the tumor volume (mm³) in response to sequential gene therapy in CT26 mouse colon cancer tumors electroporated with control (Ctr) non-expression vector followed in ten days by Ctr.

FIG. 3F illustrates the change in tumor volume (mm³) in CT26 mouse colon cancer tumors treated with 50 μg anti-WSX-1 antibody and electroporated with IL12 followed in 10 days by IL27.

FIG. 4A illustrates blood IL12 protein levels over time in mice administered by intramuscular electroporation one of two protocols: IL12 gene followed in 10 days by IL12 gene; or, IL12 gene followed in 10 days by IL27 gene. IL12 was measured from blood collected on days 1 (D1), 4 (D4), and 8 (D8) following the 1^{st} and the 2^{nd} electroporation.

FIG. 4B illustrates blood IFN-γ protein levels over time in mice administered by intramuscular electroporation one of two protocols: IL12 gene followed in 10 days by IL12 gene; or, IL-12 gene followed in 10 days by IL27 gene. IFN-γ was measured from blood collected on days 1 (D1), 4 (D4), and 8 (D8) following the 1^{st} and the 2^{nd} electroporation.

FIG. 4C illustrates blood IL27 protein levels over time in mice administered by intramuscular electroporation of one of two protocols: IL12 gene followed in 10 days by IL12 gene; or, IL12 gene followed in 10 days by IL27 gene. IL27 was measured from blood collected on day 1 (D1), 4 (D4), and 8 (D8) following the 1^{st} and the 2^{nd} electroporation.

FIG. 5A demonstrates the effects of intramuscular electroporation of pIL12 and a control (pCtr) on expression of IL27 in Balb/c mice at day 1 (D1), 4 (D4), and 8 (D8) after electroporation.

FIG. 5B demonstrates the effects of intramuscular electroporation of IL12 and a control (pCtr) on expression of IL12 in Balb/c mice at day 1 (D1), 4 (D4), and 8 (D8) after electroporation.

FIG. 5C demonstrates the effect of intramuscular electroporation of IL12 and a control (pCtr) on expression of IL27 in C3H mice at day 1 (D1), 4 (D4), and 8 (D8) after electroporation.

FIG. 6A illustrates the effects of increasing levels of recombinant IL12 protein on IL27 expression levels in Balb/c splenocytes cultured in vitro at 24, 48, and 72 hours after administering IL12.

FIG. 6B illustrates the effects of increasing levels of recombinant IL12 protein on IL27 expression levels in C3H splenocytes cultured in vitro at 24, 48, and 72 hours after administering IL12.

FIG. 6C illustrates the effects of increasing levels of recombinant IFN-γ protein on IL27 expression in C3H splenocytes cultured in vitro at 24 and 48 hours after administering IFN-γ.

FIG. 6D illustrates the effects of recombinant IFN-γ protein on IL27 expression in Balb/c splenocytes cultured in vitro at 48 hours after administering IFN-γ.

FIG. 6E illustrates the effects of recombinant IL12 protein with and without an antibody to IFN-γ; on IL27 expression in Balb/c splenocytes cultured in vitro at 48 hours after administration.

FIG. 7A illustrates the levels of IL27 expression in response to administration of recombinant IL12 protein in vitro in splenocytes from two mice strains, C57BL/6 and Balb/c, that are either wild type (NM) or knocked out for either IL12 receptor (IL12Rβ2−/−), IFN-γ receptor (IFN-γR1−/−), or IFN-γ (IFN-γ−/−) as indicated.

FIG. 7B illustrates impaired induction of IL27 expression in response to administration of recombinant IFN-γ, protein in vitro in splenocytes from two mice strains, C57BL/6 and Balb/c, that are either wild type (Wt) or knocked out for either IL12 receptor (IL12Rβ2−/−), IFN-γ receptor (IFN-γR1−/−), or IFN-γ, (IFN-γ−/−) as indicated.

FIG. 7C illustrates that knockout of Stat1 inhibits induction of IL27 expression in response to recombinant IL12 protein in splenocytes cultured in vitro from the C3H mouse strain wild type (C3H) or deficient in Stat1 (STAT1−/−).

FIG. 7D illustrates that knockout of Stat1 inhibits induction of IFN-γ expression in response to recombinant IL12 protein in splenocytes cultured in vitro from the C3H mouse strain wild type (C3H) or deficient in Stat1 (STAT1−/−).

FIG. 7E illustrates the time response of in vivo induction of IL27 expression in response to electroporated pIL12 in the C3H mouse strain wild type (C3H) or deficient in Stat1 (STAT1−/−) at day 1 (D1), 4 (D4), and 8 (D8) after IL12 administration.

FIG. 7F illustrates the time response of in vivo induction of IL27 expression in response to recombinant IFN-γ protein in the C3H mouse strain wild type (C3H) or deficient in Stat1 (STAT1−/−) at day 1 (D1), 4 (D4), and 8 (D8) after IL12 administration.

FIG. 8A indicates the tentative Stat1 binding sites based on sequence blast search on the p28 promoter, with SVM251-SVM256 representing the forward (F) and reverse (R) primer pairs flanking the Stat1 binding sites.

FIG. 8B illustrates a chromatin immunoprecipitation assay in splenocyte lysate in which Stat1 binds to putative Stat1 binding sites (˜1.1 (a), ˜1.3 (b), and ˜5 (c) kb upstream of the transcriptional start site) on the IL27 promoter in the presence or absence of IL12 and the presence or absence of an anti-IFN-γ antibody.

FIG. 8C illustrates a chromatin immunoprecipitation assay performed in splenocyte lysate in which Stat1 was found to bind to putative Stat1 binding sites (˜1.1 (a), ˜1.3 (b), and ˜5 (c) kb upstream of the transcriptional start site) on the IL27 promoter in the presence, but not absence, of IFN-γ protein.

FIG. 8D illustrates a chromatin immunoprecipitation assay in which Stat1 was found to bind to putative Stat1 binding sites (˜1.1 (a), ˜1.3 (b), and ˜5 (c) kb upstream of the transcriptional start site) on the IL27 (EB13) promoter in the presence or absence of IL12 in spleno and the presence or absence of an anti-IFN-γ antibody.

FIG. 9A demonstrates induction of IL27 expression following intramuscular electroporation of genes encoding several cytokines (pIL23, pIL15, pIL2, pIFN-α, pIFN-β, and cognate gene (pCtr)).

FIG. 9B demonstrates induction of IFN-γ expression following intramuscular electroporation of genes encoding several cytokines (pIL23, pIL15, pIL2, pIL27, and cognate gene (pCtr)).

FIG. 10A illustrates the cytolytic activity of natural killer (NK) cells as a measure of anti-tumor immunity achieved at the end of 10 days after sequential gene therapy using IL12→IL12 or IL12→IL27 in homologous tumor cells and NK cell depleted spleen cells (E:T is the ratio of effector cells to tumor cells for the standard assay for cytolytic activity.)

FIG. 10B illustrates the cytolytic activity of natural killer (NK) cells as a measure of anti-tumor immunity achieved at the end of 10 days after sequential gene therapy using IL12→IL12 or IL12→IL27 in Yac1 mouse lymphoma cells, (E:T is the ratio of effector cells to tumor cells for the standard assay for cytolytic activity.)

FIG. 10C illustrates cytotoxic T cell activity as a measure of anti-tumor immunity achieved at the end of 10 days after sequential gene therapy using IL12→IL12 or IL12→IL27 in mice. (E:T is the ratio of effector cells to tumor cells for the standard assay for cytolytic activity.)

FIG. 10D illustrates infiltration of T cells in tumors in response to IL12→IL12 or IL12→IL27 sequential gene therapy (p=0.036).

FIG. 10E illustrates the kinetics of a tumor growth measured by tumor volume (mm³) by inoculating IL12→IL12 or IL12→IL27-cured mice with a subsequent challenge of tumor cells (CT26).

FIG. 11 illustrates the effect on 4T1 tumor volume (mm³) of sequential gene therapy with IL12→IL27 administration by intramuscular electroporation in mice that were also injected with rat immunoglobin (as a control) or an antibody against one of CD8, CD4, or NK positive cell types.

FIG. 12 demonstrates differential induction of tumor-specific IFN-γ positive CD8 T cells from mice treated with either IL12→IL12 or IL12→IL27 sequential gene therapy. To ensure that effector cells were primarily CD8 positive, both NK and CD4 T cells were depleted using neutralization antibody for one day prior to euthanizing mice for cell collection.

FIG. 13 demonstrates expression of WSX-1 (part of the cognate IL27 receptor) in CD3+T, CD3−T, 4T1, and B16F10 cell lines using RT-PCR analysis.

FIG. 14 illustrates total and phosphorylated levels of Stat1 and Stat3, expression in 4T1 tumor cells grown in media alone (left lane), media supplemented with recombinant IL27 protein for 10 min (middle lane), or media+IL27 protein overnight (right lane).

FIG. 15A illustrates the reduction in mouse CT26 colon cancer tumor volume (mm³) by IL12 gene therapy using electroporation IL12 expression plasmid.

FIG. 15B illustrates the increase in aggressive CT26 mouse colon cancer tumor volume (mm³) in response to electroporation of a control non-expression plasmid.

FIG. 15C illustrates the change in tumor volume (mm³) in response to electroporation of IL12 expression plasmid in cells also administered a polyclonal against WSX-1.

FIG. 16 illustrates the efficacy of IL12→IL27 sequential gene therapy on change in volume (mm³) of tumors containing either wild type B16F10 melanoma cells or B16F10 cells stably transfected with WSX-1.

FIG. 17A illustrates functionality of WSX-1 using western blot analysis of Stat1 phosphorylation as a measure of WSX-1 activation in response to IL12→IL27 sequential gene therapy in SKBR3 human breast carcinoma cells either untreated (m) or treated with IL27 protein (4 μg) for 10 min (100′) or overnight (o/n).

FIG. 176 illustrates functionality of WSX-1 using western blot analysis of Stat1 phosphorylation as a measure of WSX-1 activation in response to IL12→IL27 sequential gene therapy in MDA231 human breast carcinoma cells either untreated (m) or treated with IL27 protein (4 μg) for 10 min (10′) or overnight (o/n).

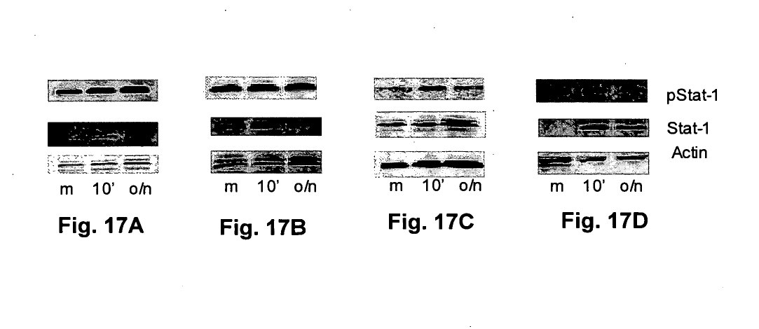

FIG. 17C illustrates functionality of WSX-1 using western blot analysis of Stat1 phosphorylation as a measure of WSX-1 activation in response to IL12→IL27 sequential gene therapy in MDA453 human breast carcinoma cells either untreated (m) or treated with IL27 protein (4 μg) for 10 min (10′) or overnight (o/n).

FIG. 17D illustrates functionality of WSX-1 using western blot analysis of Stat1 phosphorylation as a measure of WSX-1 activation in response to IL12→IL27 sequential gene therapy in Jurkat immortalized human T lymphocyte cells either untreated (m) or treated with IL27 protein (4 μg) for 10 min (10′) or overnight (o/n).

Eradication of residual malignancy and metastatic tumors via systemic approach is the key for successfully treating cancer and increasing the cancer patient survival. Systemic administration of IL12 protein in an acute large dose is effective but toxic. Systemic administration of IL12 gene by persistently expressing a low level of IL12 protein may reduce the systemic toxicity, but only eradicates IL12 sensitive tumors. Here, we discovered that sequential administration of IL12 and IL27 encoding DNA, referred to as sequential IL12-IL27 gene therapy, not only eradicated IL12 sensitive tumors from 100% of mice but also eradicated the highly malignant 4T1 tumors from 33% of treated mice in multiple independent experiments. This IL12-IL27 sequential gene therapy is not only superior to IL12-IL12 sequential gene therapy for eliminating tumors, but also for inducing CTL activity, increasing T cell infiltration into tumors, and yielding a large number of tumor-specific IFN-γ positive CD8 T cells. More importantly, the IL12-IL27 sequential gene therapy yielded a strong anti-tumor immune memory compared to IL12-IL12 gene therapy. Both reversal of the administration sequence and co-administration of IL12 and IL27 impaired the tumor eradication in 4T1 tumor bearing mice. This IL12-IL27 sequential gene therapy, via sequential administration of IL12 and IL27 encoding plasmid DNA into tumor-bearing mice through intramuscular electroporation, provides a simple but effective approach for eliminating inaccessible residual tumors.

The key for eradicating tumors by systemic IL12 gene therapy is to promote the activation of tumor-specific T cells. Our discovery indicates that sequential administration of IL12 and IL27 not only eradicated systemic tumors, but also induced a strong anti-tumor immune memory for rejecting rechallenged tumor cells. This ability to eradicate tumors and to reject tumor cells upon rechallenge was not seen in either systemic delivery of only IL12 or IL27. In this experiment the gene delivery was by intramuscular injection followed by electroporation. Other methods to introduce exogenous nucleic acid into the tumor or other organs of the mammal include the following. DEAE Dextran transfection, calcium phosphate transfection, cationic liposome fusion, protoplast fusion, creation of an in vivo electric field, DNA-coated microprojectile bombardment, injection with recombinant replication-defective viruses, homologous recombination, in vivo gene therapy, ex vivo gene therapy, viral vectors, and naked DNA transfer. These methods are well known by persons skilled in this art. See U.S. Patent Application Publication No. US 2002/0119945. This sequential therapy was more effective in tumors expressing the WSX-1 receptor.

The sequential gene therapy reported here indicated that only administration of IL12 followed by IL27 will eradicate highly malignant tumors. We discovered that sequential administration of IL-12 and IL27 encoding DNA and systemic expression of these two cytokines not only eradicates the immunogenic colon CT26 tumors in 100% of mice, but also eradicates highly malignant breast 4T1 tumors in 33% of mice. More importantly, this sequential gene therapy induces a stronger anti-tumor T cell immune response. Either reversing the administration sequence of IL12 and IL27 or co-administration of these two cytokine genes impairs the tumor eradication found by sequential administration of IL12 and IL27. This novel sequential gene therapy sheds light on developing a simple and effective systemic electroporation gene therapy for eradicating systemic microscopic malignancy.

The conventional mechanisms for IL12-mediated inhibition of tumor growth include activation of NK cells, induction of tumor-specific CD8 T cells, promotion of Th1 cell differentiation, and enhancement of IFN-γ production. This study revealed a novel mechanism, indicating that induction of IL-27 by IL12 dictates the IL12-mediated anti-tumor efficacy. This conclusion is supported by the fact that blocking IL27 receptor wsx-1 reverses IL12 gene therapy-mediated tumor eradication. In agreement with this fact, IL27 is induced by IL12 in spleen cells both in vitro and in vivo. Moreover, the IL12-mediated upregulation of IL27 is impaired in IL12Rβ2-deficient immune cells. However, this upregulation was also impaired in IFN-γ−/−, Stat1−/−, and IFN-γR−/− spleen cells, suggesting this regulation is through Stat1 and IFN-γ-dependent mechanisms. The notion of IFN-γ signaling dependence was supported by the IFN-γ-mediated induction of IL27 in spleen cells isolated from wild-type, IL12R−/− and IFN-γ−/− mice, but not from the IFN-γR−/− mice. The notion of Stat1-dependence is illustrated by the impaired induction of IL27 in Stat1−/− mice by IL12 and the Stat1-dependent binding to the IL27 promoters. This discovery suggests that many significant immune responses previously claimed by IL12 and IFN-γ may be due to the induction of IL27. This discovery also indicates that Stat1 can be used as an indicator of the presence of the WSX-1 receptor in a cell.

### EXAMPLE 1

### Materials and Methods

Gene constructs: The gene clones used in this study include IL2, IL12, IL23, IL27, IFN-α, IFN-β, and IFN-γ IL2, IL12, and IFN-α were obtained from Valentis, Inc. (Burlingame, Calif.). IL27 was from the Chiba Cancer Center Research Institute, Japan. To increase the level of gene expression, the IL27 was subcloned into the same vector as IL12, which contains a CMV promoter, a mini-intron right after the promoter, and a bGh polyadenylation signal. Murine IFN-γ was amplified from murine spleen cells by RT-PCR using the forward and backward primers (SVM147F: 5′-ATGAACGCTACACACTGCAT-3′ (SEQ ID 1) and Svm148R: 5′-TCAGCAGCGACTCCTTTC-3′ (SEQ ID 2)), which were complementary to the sequences encoding protein translation start and stop codon regions. The DNA fragment was cloned into the same expression vector as for IL12. Mouse IFN-β was amplified from mouse genomic DNA with primers Svm71F: 5′-TCATGAACAACAGGTGGATC-3′ (SEQ ID 3) and Svm144R: 5′-CAGGTCTTCAGTTTTGGAAG-3′ (SEQ ID 4), because this murine gene encoding region does not contain any intron. Likewise, IFN-β was cloned into the same expression vector as IL12. The p19 subunit was amplified using primers UAMS129: 5′-atgctggaftgcagagcagt-3′ (SEQ ID 5) and UAMS130: 5′-tgggcatccttaagctgttg-3′ (SEQ ID 6). The amplified DNA fragment encoding p19 was cloned into the TA2.1 vector and sub-cloned into IL12 encoding expression vector to replace the p35 encoding subunit, yielding IL23 gene construct. All the new clones were confirmed by sequence analysis and the biological function. Plasmid DNA was manufactured using Endotoxic-free Mega preparation kit from Qiagen, Inc. (Valencia, Calif.) following the manufacturer's instructions.

Tumor models and DNA delivery via intramuscular electroporation. Six- to eight-week-old female C3H/HeN and Balb/c mice, weighing 18-20 g, from the in-house animal breeding facility were used for this study and were maintained under National Institutes of Health guidelines. All the animal procedures including tumor transplantation, tumor volume monitoring, gene administration, bleeding and mice euthanization were approved by the Institutional Animal Care and Use Committee (IACUC) of Louisiana State University.

Cell lines and cell culture: SCCVII are spontaneously arising murine squamous cell carcinomas; 4T1 is a mouse adenocarcinoma mammary tumor cell line; CT26 is a mouse colon adenocarcinoma; SKBR3 is a human mammary carcinoma; MDA231 is a human mammary adenocarcionma; MDA435 is a human mammary carcinoma; Jurkat cells are immortalized T lymphocytes, and Yac1 cells are mouse lymphoma (available from ATCC, Manassas, Va.) The cell lines were maintained in Dulbecco's modified Eagle's medium (DMEM) containing 10% fetal bovine serum (Life Technologies, Rockville, Md.).

Xenograft models: Tumors were generated by subcutaneously inoculating the mice. The subcutaneous tumor model was generated by subcutaneously inoculating CT26 colon tumor cells (2×10⁵ in a 30-μL volume per mouse) into Balb/C mice. The adenocarcinoma 4T1 model was generated by subcutaneously inoculating 4T1 tumor cells (1×10⁵ in a 30-μL per mouse) into Balb/C mice. Both tumor cell lines were maintained in Dulbecco's modified Eagle's medium (DMEM) containing 10% fetal bovine serum (Life Technologies, Rockville, Md.). Tumor dimensions were measured with calipers every 3 days, and the volume was calculated from the formula. V=(π/8)(a×b²), where V=tumor volume, a=maximum tumor diameter, and b=diameter at 900 to ‘a’.

Using the protocols described previously, IL12 and control plasmid DNA (10 μg in a volume of 30 μl per mouse) were injected into muscles via electroporation (4). The electroporation parameters for intramuscular injection are 350 V/cm and 20 ms pulse duration for 2 pulses (14). The hind limb tibialis muscles were used for DNA administration via electroporation and a total of two administrations were performed once every 10 days. The cognate gene product could be transcribed and translated from the injected gene in the muscles and secreted into blood circulation. Tumor growth and tumor eradication were monitored every 3 days. The indicated rabbit polyclonal antibodies were administered into mice (50 μg per mouse once every three days) via intraperitoneal administration.

The mouse strains Balb/c C3H, and C3HStat1−/− were also administered with the plasmid indicated on each figure via intramuscular electroporation to determine the gene expression in serum. Blood was obtained via a cheek bleeding method—tightly grabbing the mouse neck and poking the skin on the cheek with a 16 gauge needles the bleeding will stop immediately upon releasing the fingers from the mouse neck. Blood was collected in the indicated time in the figures and serum was separated from the coagulated blood cells by centrifugation at 200×g. Serum was used for detecting IL12, IFN-γ and IL27 expression using ELISA kits purchased from R&D system (Minneapolis, Minn.). Mice survival curves (Kaplan-Meier) were drawn using Statistical software.

Expression and activation of Stat1: Western blot analysis was used to determine Stat1 activation using the antibodies anti-Stat1, anti-Stat3, and anti-phosphorylated Stat1 and anti-phosphorylated Stat3 (all obtained from Upstate Cell Signaling Solutions, Inc., Charlottesville, Va.). Tumor samples were collected on day 1 after the administration of either the gene or drug. The Western blot protocol was as described previously (16), and the chemiluminescent signal was captured using a Kodak 410 imager (Perkin Elmer, Shelton, Conn.).

In vitro treatment with rIL12 and rIFN-γ. Spleen cells were derived from 3-4 mice for each in vitro experiment using the same preparation procedure as for CTL (20). Spleen cells were seeded in 12-well pates at a cell density of 1×10⁷ cells per well in 1-2 mL of complete RPMI growth medium. The proper amount of rIL12 and rIFN-γ proteins, purchased from R&D System, was added into each well to make a final concentration as indicated in each figure. PBS was used to dilute the recombinant cytokine proteins and therefore, the same volume of PBS was added in the control wells.

Analysis of IL12, IL27 and IFN-γ expression: The expression of IL12, IL27, and IFN-γ in tumors was determined using the corresponding ELISA analysis kits from R&D Systems (Minneapolis, Minn.). Tissues for ELISA were obtained 3 days after intratumoral (tumor-local) administration of plasmid DNA via electric pulses. Each collected serum sample (50 μl per mouse) was transferred into a single well of each cytokine assay plate supplied by the manufacturer (R&D System), followed by washing and binding with the primary and the secondary antibodies. The secondary antibody contains HRP enzyme that can metabolize the substrate to release green color. The color changes to yellow-brown upon addition of the stop reaction buffer. The color intensity representing the level of gene expression was determined in the plate reader at 405 nm. A column of standard for each cytokine (ranging from 0 to 500 pg per well in a 2-fold escalated dilution) was set in each assay plate to convert the light absorbance from each sample into the weight (pg) per mL of serum. All the reagents were included in each ELISA kit purchased from the manufacturer (R&D System).

RT-PCR for WSX-1. RNA was isolated from tumor cells using Trizol reagent (Invitrogen). 10 μg of total RNA was treated with DNA-free™ DNase Treatment and Removal Reagent (Ambion Inc. Austin, Tex.). Two μg of DNA-free RNA was converted to cDNA using High Capacity cDNA Archive Kit (Applied Biosystems, Foster City, Calif.) in a 20 μl reaction. For amplifying WSX-1 the following pair of mWSX-1 primers were used.

| | |
|---|---|
| SVM177: | |
| 5′- CAA GAA GAG GTC CCG TGC TG-3′ | (SEQ ID 7) |
| SVM178: | |
| 5′- TTG AGC CCA GTC CAC CAC AT -3′ | (SEQ ID 8) |

Four μl of the cDNA was used for amplifying WSX-1. The PCR program was as follows: 1 cycle of 95° C., 3 min, 40 cycle of 95° C., 30 second, 60° C. 1 min. PCR product size was 237 bp.

To amplify the house keeping gene beta-Actin, the primers shown below were used. 1.16 μl of the same cDNA used for amplifying WSX-1 was used for beta actin amplification in a 20 μl reaction. PCR program was as follows: 1 cycle of 95° C., 3 min, 33 cycle of 95° C., 30 second, 65° C. 1 min. PCR product size: 540 bp.

| | |
|---|---|
| Forward: | |
| 5′- GTG GGC CGC CCT AGG CAC CAG-3′ | (SEQ ID 9) |
| Reverse: | |
| 5′- TCT TTG ATG TCA CGC ACG ATT TC-3′ | (SEQ ID 10) |

Statistical Analysis. A two-sided Student's t-test was used to compare the means of individual treatments. P values less than 0.05 were considered statistically significant.

Generation of WSX-1 recombinant protein: Mouse wsx-1 construct was purchased from Open Biosystems (Huntsville, Ala.). Wsx-1 full length was amplified from wsx-1 clone purchased from Open Biosystems (Huntsville, Ala.) via polymerase chain reaction (PCR) using forward and reverse primers Svm198F: 5′-CGGATCCATGAACCGGCTCCGGGTT-3′ (SEQ ID 11) and SVM199R: 5′-TCAGACTAGAAGGCCCAGCTC-3′ (SEQ ID 12), respectively. The amplified DNA fragment was cloned into pRSETA which is a vector for expressing recombinant protein from bacteria host. Wsx-1 was expressed in bacterial host BL21 DE3 plysS and purified using ProBond Purification System under denaturing conditions (Invitrogen, Carisbad, Calif.).

Generation of WSX-1 polyclonal antibody: The column purified WSX-1 protein was further purified with SDA-PAGE to remove any contaminated proteins. The SDS-PAGE-separated WSX-1 protein was sliced off the gel using a sharp razor blade, and the sliced protein-gel was homogenized by a bead-beater for 5 minutes at the maximum speed to become get slurry. Prior to immunizing the rabbits (2.5 kg per rabbit) with the homogenized protein-gel slurry by subcutaneous injection, 100 μg homogenized protein-gel slurry was mixed with 1 mL Complete Freund adjuvant by syringe thoroughly. Two additional injections (the second and third boosts) were performed on weeks 4 and 5 after the initial priming. The Incomplete Freund adjuvant was used to mix with the same amount of WSX-1 protein gel homogenate for each of the two boost injections. One week after the third immunization, blood was collected and Western blot analysis was performed to confirm the antibody binds to WSX-1. After the confirmation, blood was collected on day 10 after the final boost for purifying the anti-WSX-1 antibody from the serum. Wsx-1 antibody was purified with Sepharose protein-A column or by Pacific Immunology Co. (Ramona, Calif.). The purified WSX-1 antibody was used for blocking IL27 signaling by binding with WSX-1, which was achieved by administering 50 ug antibody per mouse. This dose was selected because the IL27 gene therapy-induced Stat3 phosphorylation in spleen cells was effectively inhibited in vivo by administering 50 μg antibody per mouse. (Data not shown)

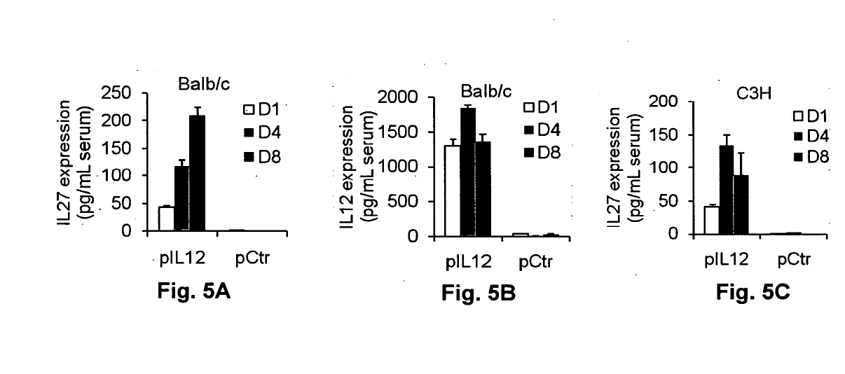

CHIP assay. The procedure for this assay is the same as the protocol from Upstate Biotechnology (Lake Placid, N.Y.). In brief, splenocytes were prepared as described previously for CTL assay (20). Spleen cells (1×10⁷ per sample) were incubated with or without rIL12 protein at a concentration of 1 ng/mL in 3 mL heat-inactivated 10% serum-containing RPMI media. Following 48 h treatment, histones were cross-linked to DNA by adding 1 mL of 4% formaldehyde directly to 3 mL culture medium to a final concentration of 1% and incubating for 10 min at 37-C. Cells were lysed with SDS Lysis Buffer (200 μl per 1×10⁶ cells) and lysate was sonicated to shear genomic DNA into fragments between 200 to 500 bp using Branson Sonifier 450 at the parameters of 30 s, 10 cycles, output 5, and duty cycles 45%. The Stat1 binding DNA was precipitated with 2 μg pStat1 polyclonal antibody (Santa Cruz, Inc., Santa Cruz Calif.) and 60 μL Protein-A agarose/Salmon Sperm DNA. To determine whether the Stat1 DNA binding fragment is precipitated, the DNA was eluted from the immunoprecipitation complex with elution buffer (1% SDS, 0.1 M NaHCO₃) and released from the histone-DNA cross-linked complex by heating at 6500. The released DNA was isolated by phenol/chloroform extraction and ethanol precipitation. DNA was re-suspended in sterilized water and PCR was performed to detect the Stat1 binding DNA fragments. The forwarding (F) and reversing (R) primer sequences used for PCR detection of the various Stat1 binding sites (a, b, and c) are shown in FIG. 5A. These bound DNA fragments were referred to as SVM251-256. To determine that an equal amount of DNA was used, the input DNA from each sample was extracted and quantitated using the same PCP methods.

Immunostaining of T cell infiltration and CTL activity assay: Immunostaining of T cell infiltration was performed on frozen tumor sections. The procedures for frozen-block preparation, tissue sectioning, and immunostaining were the same as described previously (4, 5, 8, 14, and 16). Tumor samples were collected 10 days after the final administration to evaluate the T cell numbers via immunostaining. The primary antibody applied to the sections was anti-COB (1:400, Santa Cruz Biotechnology, Santa Cruz, Calif.).

The same fluorescence-based CTL assay method as described previously was used to determine tumor-specific T and NK cell cytolytic activity against target tumor cells (13). To determine NK cell cytolytic activity in vitro, T cells were depleted by administering both anti-CD4 (GK1.5) and -CD8 T cell antibodies (50 μg per mouse) into the sequential gene therapy-treated mice via intraperitoneal injection one day prior to euthanizing mice. To determine the cytolytic activity of T cells against tumor cells in vitro, NK cell-depleted spleen cells were used.

ELISPOT assay to determine the tumor-specific IFN-γ positive CD8 Tcells from the cured mice: To determine the acute induction of tumor specific CD8 T cells by tumor antigen stimulation, the IL12→IL27 sequential gene therapy-cured mice were challenged with tumor cells for 3 days prior to euthanization for analysis of tumor-reactive IFN-γ-secreting CD8 T cells. To avoid the effect of IFN-γ-secreting NK and CD4 T cells on accurately determining the number of IFN-γ positive CD8 T cells, mice were administered with anti-NK1.1 (PK136) and anti-CD4 T cell antibodies (GK1.5) one day prior to euthanizing mice. Lymphocyte cells were isolated from Lymph nodes by smearing the tissue and pushing through a 70 μm strainer. A total of 50 μg per mouse was administered by intraperitoneal injection, and flow cytometry was performed to confirm that the primary collected cells were CD8 T cells the next day when mice were euthanized. The isolated CD8 T cells were incubated without and with mitomycin C-treated target tumor cells (CT26) in the IFN-γ capturing ELISPOT plate purchased from R&D Systems. After incubating overnight, the plate washed and the IFN-γ spots were detected using immunostaining following the manufactures instruction. The image of IFN-γ positive spots was captured via Kodak image station 440 (Rochester, N.Y.).

Statistical Analysis of tumor volume CD8⁺ T cell infiltration, and gene expression. The two-sided Student's t test was used to compare individual treatments. Survival analysis was performed with the Chi-square analysis. P values less than 0.05 were considered statistically significant.

### EXAMPLE 2

### Sequential Gene Therapy Using IL12 and IL27

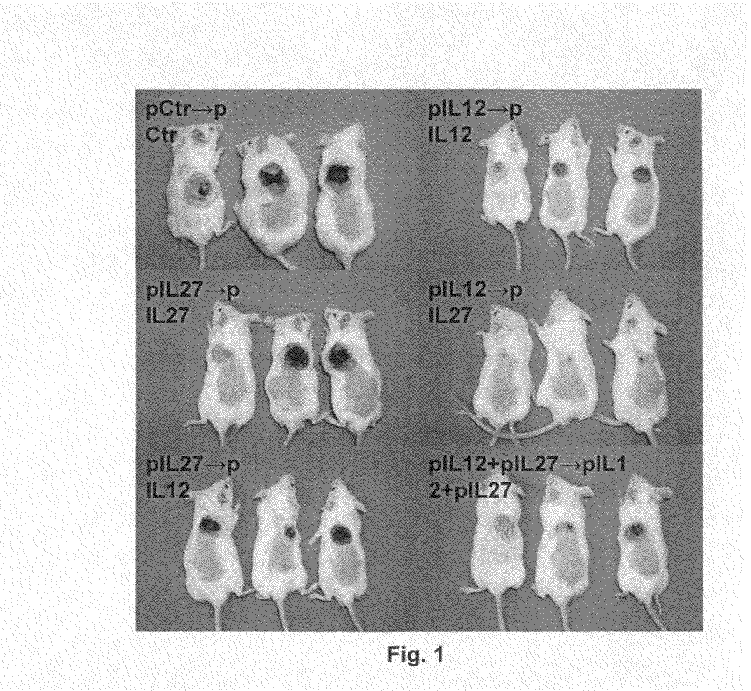

Balb/c mice were inoculated with 4T1 tumor cells. The 4T1 tumor model was chosen because this model is a highly malignant tumor, equaling the clinical grade IV malignancy of breast cancer (11). Within 4-5 days, tumors reached approximately 3-4 mm in diameter. The mice were then given the indicated plasmid DNA (IL12, IL27, or control) with a syringe followed by intramuscular electroporation. The mice were given one administration of 10 μg DNA at day 5 and a second one at day 15 after inoculation of tumor cells. The various treatments were as follows: control plasmid (pCtr) followed by a second pCtr (control); plasmid with IL27 gene (IL27) followed by same plasmid (IL27) plasmid with IL12 gene (IL12) followed by the same plasmid (IL12); plasmid with IL12 gene (IL12) followed by plasmid with IL27 gene (IL27); plasmid with IL27 gene (IL27) followed by plasmid with IL12 gene (IL12); and a combination of plasmids with IL12 gene and with IL27 gene combination followed by the same combination. Tumor volumes were measured every 3 days after beginning treatment. FIG. 1 is a photograph of the various groups of three mice at the end of 18 days after the administration of the second DNA. As shown in FIG. 1 the most effective treatment was the sequential treatment of IL12 followed by IL27. This treatment completely eradicated the highly malignant 4T1 tumors located distantly from the injected muscles. The reversed administration failed to eradicate tumors, as did the co-administration of both gene-encoding plasmid DNA. Neither IL12 nor IL27 treatment alone was successful in eradicating tumors.

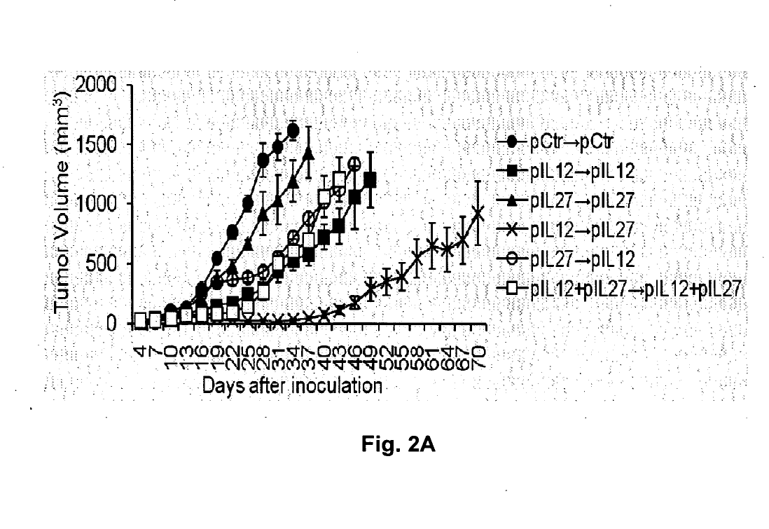

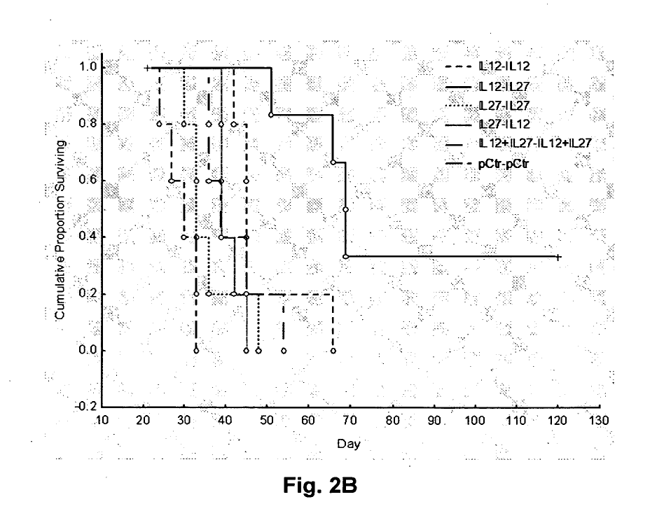

Sequential gene therapy by electroporation reduced tumor volumes using only 10 μg plasmid DNA encoding IL12 followed by 10 μg plasmid DNA encoding IL27 for each mouse. Using the same protocol as described above in 5 mice, FIG. 2A shows the reduction in tumor volume. (FIG. 2A). In addition, gene therapy with IL12 increased survival time as indicated in FIG. 2B which shows a Kaplan-Meier survival curve for the mice pictured in FIG. 1 in which 33% of mice were cured of tumors. The curves for IL12→IL IL12→IL27 are significantly different (p<0.05). These tumor-eradicated mice from the IL12→IL27 treatment remained tumor free during the two-month observation period. After 2 months, these tumor-eradicated (“cured”) mice were challenged with the same homologous 4T1 tumor cells. Significantly, 83% of the cured mice (5 out of 6) rejected the 4T1 tumor cells. This illustrated that a long term anti-tumor immune memory had developed and indicates that a primary function of IL27 is to enhance the generation of tumor-specific immune memory T cells that may be initiated by the IL12 treatment.

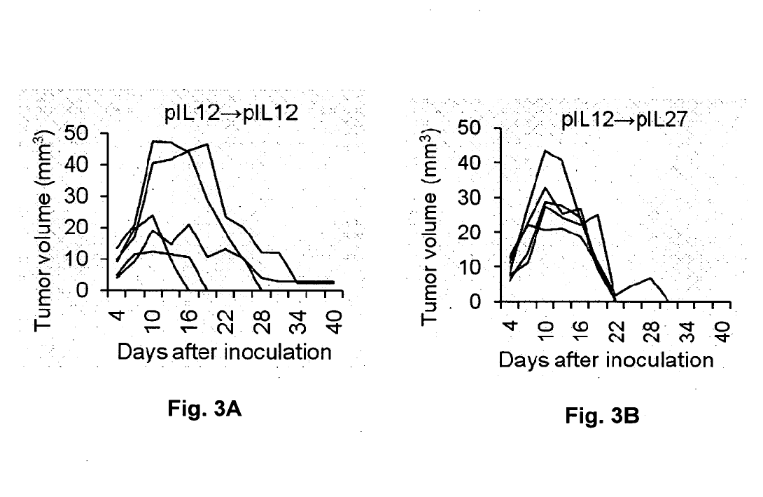

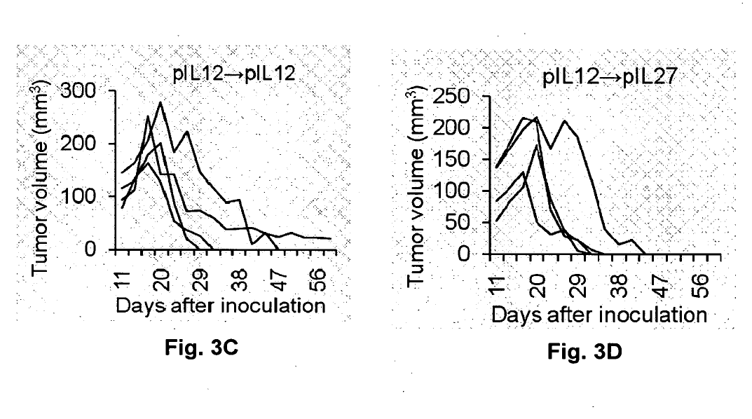

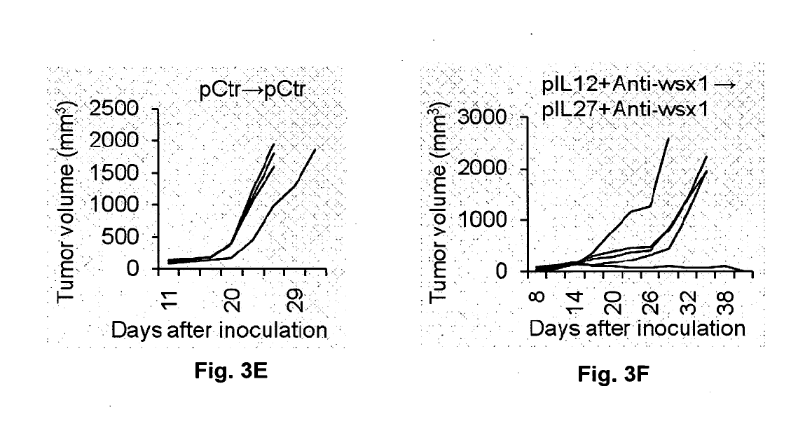

CT26 colon tumors are known to be immunogenic and sensitive to IL12 therapy. Using the same protocol as described above for FIG. 1, muscle-based sequential administrations of IL12 encoding plasmid DNA alone with a 10-day interval caused eradication of 60-75% of tumors from two independent experiments at the observed period (FIGS. 3A and 3C). This treatment is referred to as IL12→IL12 sequential gene therapy in which IL12 plasmid DNA is given in both administrations. Compared to IL12→IL12 sequential gene therapy, IL12→IL27 sequential gene therapy is more effective because tumors were eradicated in 100% of CT26 tumor-bearing mice from two independent experiments with an initial tumor diameter as large as 5-7 mm (FIGS. 3B and 3D). FIG. 5E indicates the aggressive tumor growth when only the control plasmid DNA is administered. These results indicate that exogenous administration of IL27 at a later point after IL12 treatment can enhance anti-tumor activity even in the IL12 sensitive tumor model. Administration of an antibody that binds to the IL27 receptor, WSX-1, reversed IL12→IL27 sequential gene therapy-caused tumor eradication in 80% of the mice (FIG. 3F), and resulted in aggressive tumor growth as observed in the control group receiving control DNA (FIG. 3E), further illustrating the significance of the exogenous IL27 in causing tumor regression.

### EXAMPLE 3

### Cytokine Expression Profiles Using IL12 and IL27 Gene Therapy

Using various sequential gene therapy protocols administered to tumor-injected mice, serum concentrations of IL12, IL27, and IFN-γ were assayed over time. Each mouse was injected with 10 μg DNA via intramuscular electroporation at each administration. The sequential gene protocols were basically as described above, and included the following: IL12 gene followed by IL12 gene and IL12 gene followed by IL27 gene. The first gene administration was on day 5 after inoculation with tumor cells, and the second gene administration on day 15. Blood was collected on days 1 (D1), 4 (D4), and 8 (D8) after the first (1^{st}) and the second (2^{nd}) administration of DNA. An ELISA kit was used to assay the concentration of each cytokine expression.

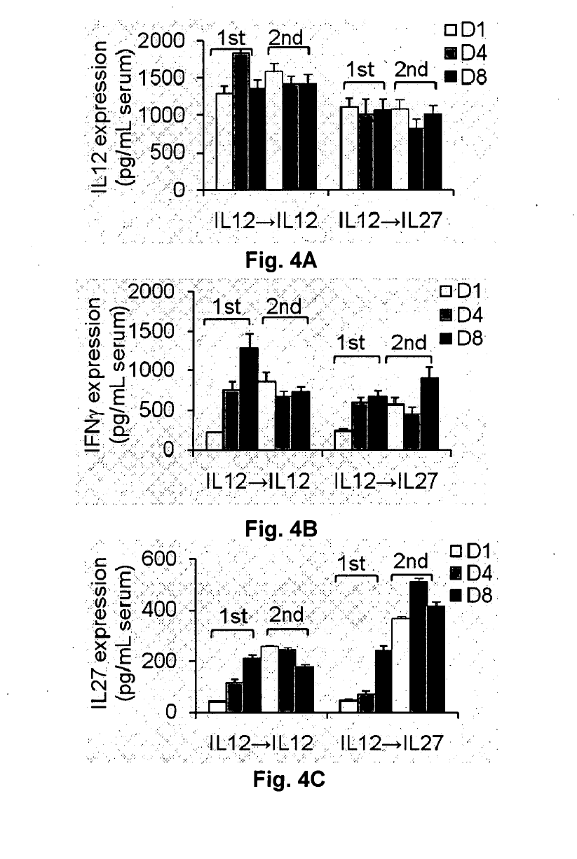

One possible mechanism for the enhanced tumor eradication by the IL12-IL27 sequential gene therapy is the synergistic induction of IFN-γ. To test this assumption, the expression of IFN-γ on the indicated dates after the first and the second administration was compared between the two treatment groups. IL12-IL12 vs. IL11-IL27 gene therapy. No enhanced expression of IFN-γ was found in the IL12-IL27 treatment group (FIG. 4B) and similar levels of IL12 and IFN-γ expression were found in these two treatment groups (FIGS. 4A, 4B). As anticipated, an increased level of IL27 in the later phase was detected by the IL12-IL27 gene therapy after administration of the exogenous IL27 encoding DNA (FIG. 4C). This result suggests that the increased IL27 expression by IL12-IL27 sequential therapy may directly contribute to the observed 4T1 tumor regression which was not obtained by the IL12-IL12 gene therapy in this model (FIG. 1). This notion is supported by the fact that administration of IL27 receptor-blocking antibody impaired tumor eradication by this sequential treatment (FIG. 3F).

Sequential therapy of IL12-IL27 did not upregulate IL12 expression but did cause significant induction of IFN-γ expression in vivo (FIGS. 4A and 4B). As shown above in Example 2, systemic expression of IL27 via intramuscular electroporation barely inhibited tumor growth. Co-administration of IL12 and IL27 was not as effective as IL12 alone in inhibiting tumor growth. These observations together indicate that expression of IL12 and upregulation of IFN-γ is a requirement for tumor eradication in the first phase, and that IL27 may not be needed during this initial phase for inducing anti-tumor immune cells. However, in the second phase, for tumor eradication, a higher level of IL27 expression is required because administration of IL27 10 days after IL12 treatment eradicated the tumors (FIGS. 1 and 2A) and induced a long term anti-tumor immune memory.

Eradication of highly malignant 4T1 tumors by sequential IL12 and IL27 gene therapies by intramuscular electroporation was an unexpected result. Neither co-administration of IL12 and IL27 nor sequential administration of IL27 and IL12 (a reversed order of IL12 and IL27) eradicated the 4T1 tumor (FIG. 1). This result indicates that IL12 and IL27 are needed at specific times to effectively cause tumor regression and to induce anti-tumor immune memory.

### EXAMPLE 4

### IL12 Induces IL27 Expression in Vitro and in Vivo

The levels of circulating cytokines in the blood in response to gene therapy with IL12 were analyzed. II12-encoding plasmid DNA was administered into Balb/c and C3H mice (n=4) via intramuscular inject followed by electric pulse to enhance the DNA uptake by muscle cells. pCtr and pil12 represent control and IL12 encoding plasmid DNA in FIGS. 5A-5C. The gene product IL12, expressed in the injected muscles, was secreted into the blood circulation. Blood was collected from each mouse on days 1 (D1), 4 (D4), and 8 (D8) after administration. It was found that muscle electroporation of IL12 caused an persistent increase in IL27 expression at a maximum level of 200 pg per mL blood that peaked after 8 days (FIG. 5A). This induction was IL12 gene specific because administration of control plasmid DNA failed to induce any IL27 expression (FIG. 5A). To further illustrate that the upregulation of IL27 by administering IL12-encoding plasmid DAN is dependent on the expression of IL12, the level of IL12 was determined in the blood samples collected from Balb/c mice that received IL12-encoding DNA and control plasmid DNA. The expression of IL12 was only detected in the mice that received IL12 gene, and not in the mice receiving control plasmid DNA (FIG. 5B).

To further support this unique observation, C3H mice were also injected with the IL12 gene as described above. As found in the Balb/c mice, a high level of induction of IL27 by IL12 was detected in the C3H mouse strain (FIG. 5C).

### EXAMPLE 5

### Role of IFN-γ and Stat1 in IL12-Mediated Induction of IL27

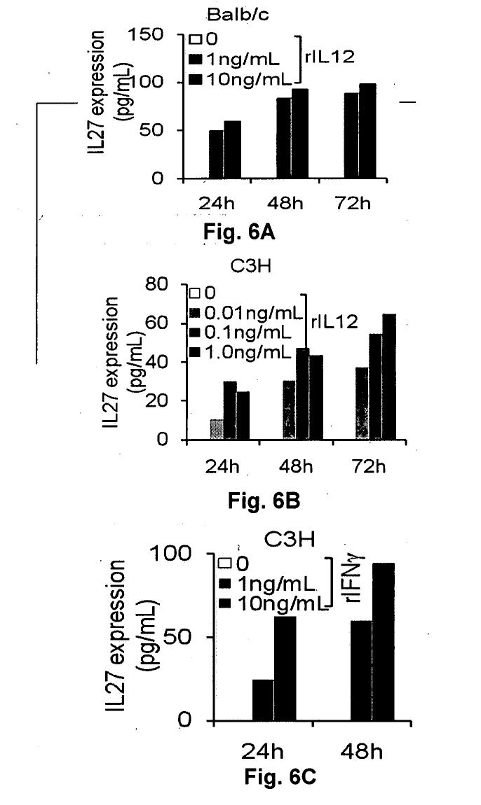

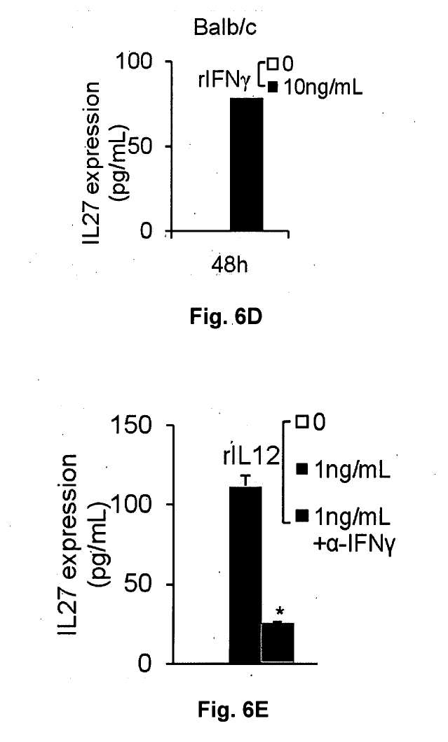

To understand the mechanism by which IL12 induces IL27 expression, in vitro treatments with and without recombinant IL12 or IFN-γ protein (rIL12 and rIFN-γ) were performed using naïve spleen cells. For these in vitro studies, rIL12 was used because DNA transfer in vitro could cause the induction of other inflammatory cytokines that could affect the results. The in vitro results repeated the in vivo observations because addition of rIL12 to the naïve spleen cells induced IL27 expression in a dose- and time-dependent fashion (FIG. 6A). This observation was independently confirmed in spleen cells isolated from both Balb/c and C3H mice (FIGS. 6A and 6B). This induction was very sensitive because rIL12 protein at a concentration of 10 pg/mL induced a 30-fold upregulation of IL27 expression in the spleen cells within 48 hours (FIG. 6B). This sensitive and high magnitude of induction by rIL12 protein in vitro further confirms the in vivo observation.

To understand the mechanism of IL12-mediated IL27 induction, whether the IL12-induced IL27 expression occurs directly from IL12 signaling or indirectly from the IL12-induced effector molecule was initially determined. One hallmark effector molecule that is induced by IL12 is IFN-γ, and previously it was known that administration of the IL12 gene via the intramuscular injection followed by electric pulses induced a significant level of IFN-γ expression. To determine whether IL27 was induced by IL12-induced IFN-γ, splenocytes were isolated from naïve mice and were subjected to the treatments with rIFN-γ. The same as rIL12 (FIGS. 5A, 6B), rIFN-γ protein induced IL27 expression in spleen cells isolated from both mouse strains (FIGS. 6C, 6D). To further illustrate that IL12 induces IL27 through the induction of IFN-γ, IFN-γ neutralization antibody was added into rIL12-treated spleen cells. Impairment of IL12-induced IL27 expression by adding IFN-γ neutralization antibody would suggest that IFN-γ is the direct effector molecule that regulates IL12-induced IL27 expression. As anticipated, IL12-induced IL27 expression was greatly impaired in the presence of IFN-γ neutralization antibody (FIG. 6E).

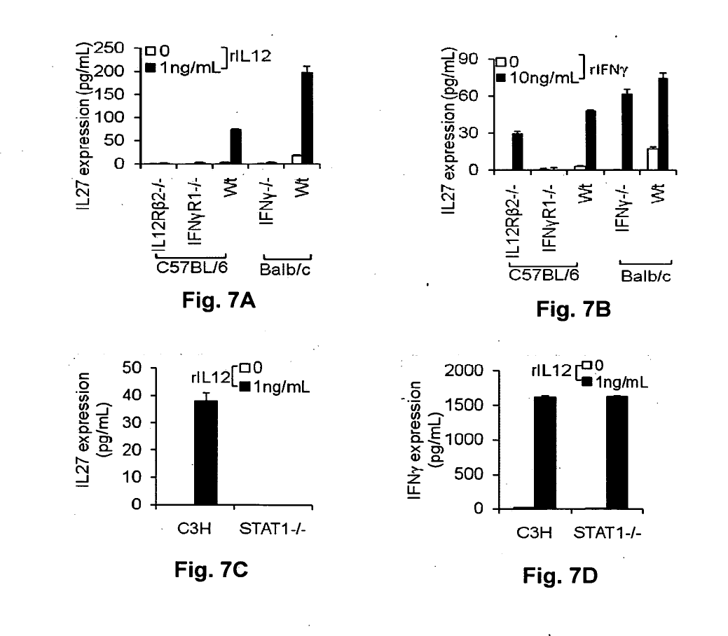

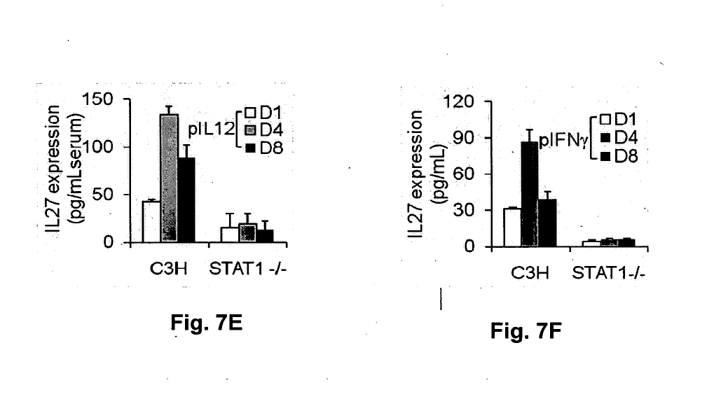

The above result indicates that IFN-γ was the key effector molecule for the IL12-mediated IL27 induction, but did not exclude the possibility that both IL12 and IL12-induced IFN-γ independently induced IL27 expression. To test this assumption the spleen cells deficient for IL12Rβ2, IFN-γ, or IFN-γR were treated with rIL12 or rIFN-γ. Impairment of IL12-induced IL27 expression in IFN-γ and IFN-γR deficient spleen cells, and impairment of IFN-γ-induced IL27 expression in IFN-γR-deficient cells would suggest that IFN-γ, but not IL12, is a direct effector molecule in inducing IL27. Indeed, IL12 failed to induce IL27 expression in the spleen cells deficient in not only IL12Rβ2 but also in IFN-γ and IFN-γR expression (FIG. 7A), suggesting that IL12 is not the direct inducer of IL27.

In the same gene deficient spleen cells, rIFN-γ induces IL27 expression in both IL12Rβ2 and IFN-γ deficient spleen cells and only failed to induce any expression in IFN-γR deficient spleen cells (FIG. 7B). This result strongly suggests that IFN-γ is the direct inducer of IL27, and the IL12-mediated IL27 induction is exclusively dependent on IFN-γ signaling. If this conclusion is true, then the IL12-mediated IL27 induction would be impaired in Stat1 deficient mice, since IFN-γ signaling is primarily dependent on Stat1. To test this assumption, spleen cells that are Stat1 deficient were treated with rIL12 (“recombinant IL12”) protein. As anticipated, rIL12 treatment failed to induce any IL27 in the Stat1 deficient spleen cells in vitro (FIG. 7C), but induced an equal amount of IFN-γ between Stat1 deficient and wild-type spleen cells (FIG. 7D). The same as found in vitro, systemic expression of either IL12 or IFN-γ via intramuscular gene therapy approach failed to induce IL27 expression in the Stat1 deficient mice in vivo (FIGS. 7E, 7F).

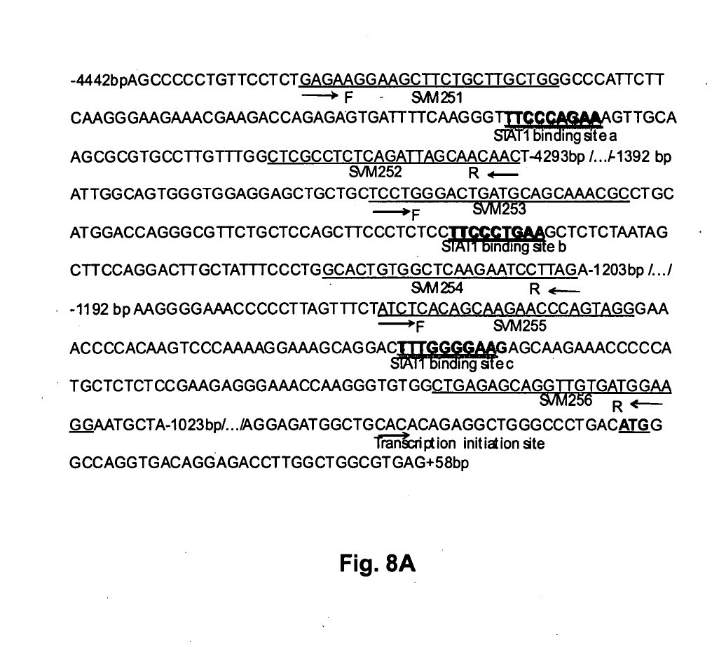

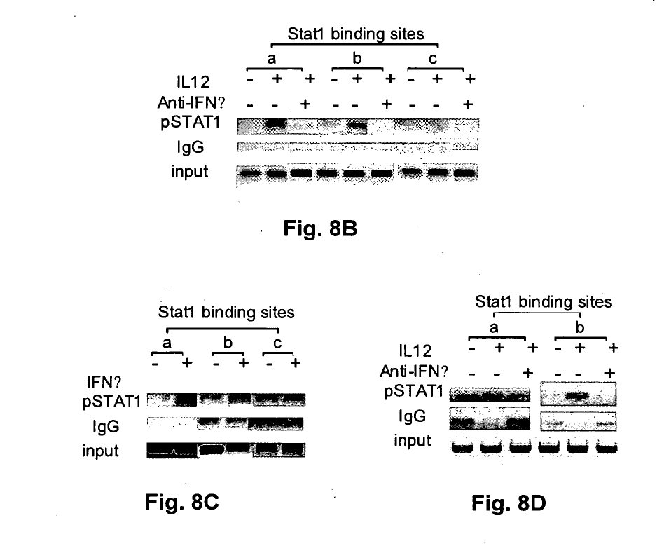

To confirm the induction of IL27 by IFN-γ is Stat1-dependent at a molecular level, chromosome immunoprecipitation (ChIP) assay was performed to determine whether rIL12 treatment induces Stat1 binding on the IL27 promoter. Both IL27 subunits EBI3 and p28 promoters contain putative GAS (“gamma activated sequence”) sites for Stat1 binding, and both promoters were tested with the ChIP assay. In the p28 promoter, the three Stat1 binding sites are located in the distal (5 kb upstream of the transcription starting site) and proximal regions (−1.1 and 1.3 kb upstream of the transcription starting site) (FIG. 8A). Two out of the three binding sites, one proximal and one distal Stat1 binding site (binding site ‘a’ and ‘b’, respectively, see FIG. 8A) on the p28 promoter, bound Stat1 in an IL12 treatment-dependent manner (FIG. 8B). These two binding sites share 96 and 92% percent homology with the known Stat1 binding sequence GAS, and the failed binding site contains only 86% homology with GAS. Therefore, the detected binding activity correlated to the level of homology with GAS. This binding result further supports the notion that the IL12-mediated IL27 expression is Stat1-dependent. The same Stat1 binding activity on p28 promoter was observed after IFN-γ treatment (FIG. 8C). The Stat1 binding activity was also clearly detected in the proximal binding site of EBI3 promoter and was weakly found in the distal binding site (FIG. 8D, promoter map is not shown). The binding activity was impaired by addition of anti-IFN-t antibody (FIG. 5D), indicating that IL12-induced Stat1 binding is IFN-γ dependent.

### EXAMPLE 6

### Induction of IL27 by Other IFN-γ-Inducing Cytokine Genes

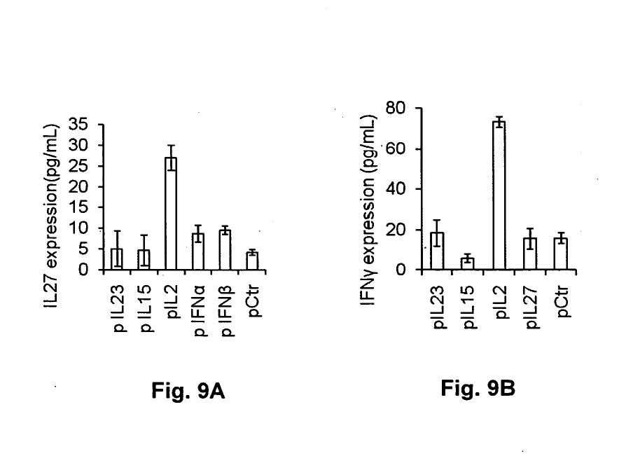

Because IFN-γ can be induced by other cytokines, whether known IFN-γ producing cytokines could also induce IL27 was analyzed. DNA encoding cytokine genes was administered via intramuscular electroporation. Serum was collected 3 days after the treatment. pCtrl, pIL2, pIL15, pIL23, pIFN-α, and pIFN-β represent control DNA and the cognate gene encoding plasmid DNA. Administration of the IFN-γ-inducing gene IL2 via intramuscular electroporation also induced expression of IL27 (FIG. 9A). IFN-γ is referred to as type II IFN, so type I IFNs (IFN-α and IFN-β) were also used to see if they induced IL27. As shown in FIG. 9A, neither IFN-α nor IFN-β induced IL27 expression. The magnitude of IL27 induction is associated with the level of IFN-γ induction by other cytokines in vivo (FIG. 9A vs. 9B). Unlike IL12, IL23 did not trigger a high level of IL27 expression (FIG. 9A vs. 9B).

### EXAMPLE 7

### Recruitment of Primary Immune Cells to IL12 Therapy

One of the main IL12 functions is to activate NK cells, inducing NK cell-mediated tumor cell death. Here, the cytolytic activities of NK cells, isolated from both IL12→IL12- and IL12→IL27-treated mice, were compared against non-specific tumor target cells. The same dose, treatment schedule and sequence described above for Example 2 (FIG. 1) were used in the experiment. Ten days after the final (second) administration, anti-tumor immune responses between the two treatments were analyzed.

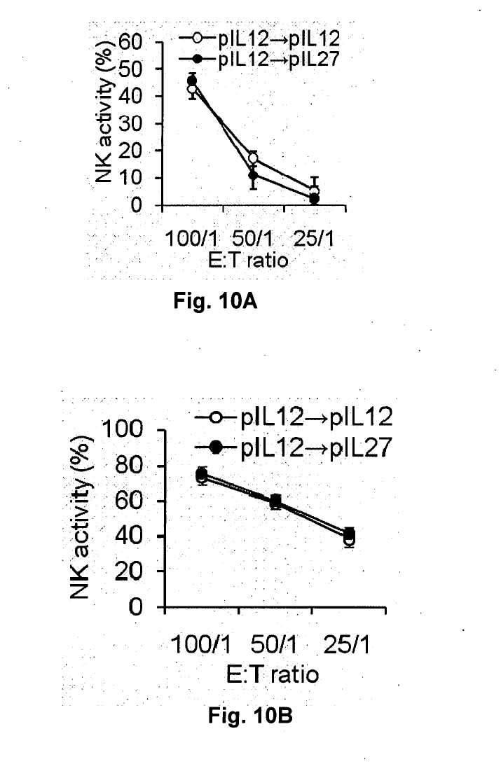

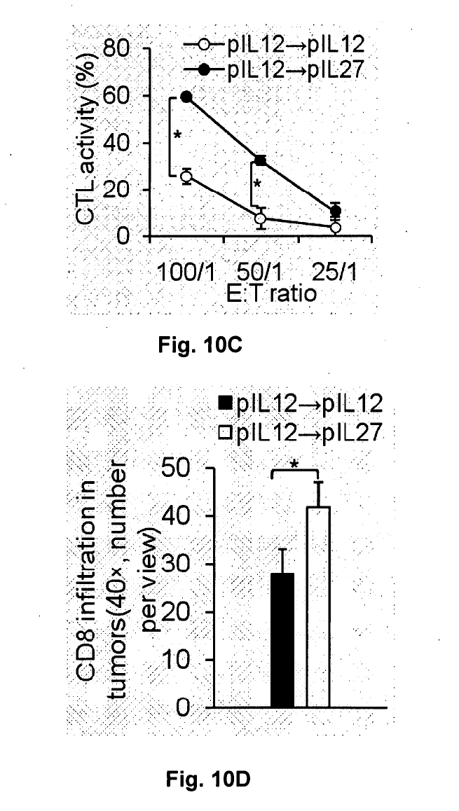

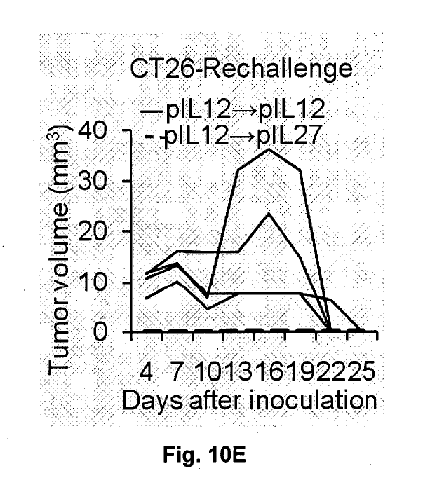

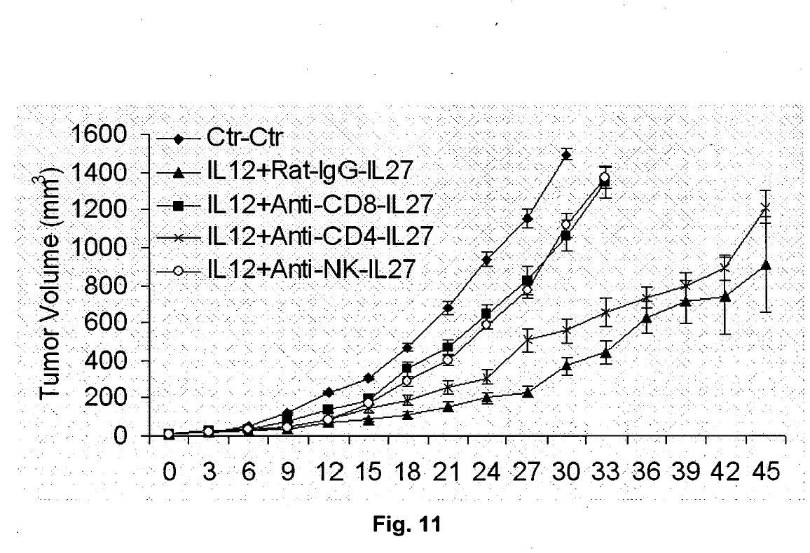

To avoid potential complications of the effects of T cells, T cells were depleted using anti-CD8 antibody one day prior to euthanizing mice. A similar level of NK cell activity (using homologous tumor cells and NK cell depleted spleen cells) was detected from both IL12→IL12 and IL12→IL27 therapies, suggesting NK activity is not the cause of different antitumor activities in these two groups (FIGS. 10A and 10B).

To test the T cell response by the sequential IL12→IL27 gene therapy, the tumor specific CTL activity was determined. A much higher level of CTL activity was detected from the IL12→IL27 treated mice, compared to IL12→IL12 treated mice (FIG. 10C). Moreover, IL12→IL27 sequential gene therapy also induced more infiltration of CD8T cells into tumors than did the IL12→IL12 treatment (FIG. 10D). These data suggest that the successive combination IL12→IL27 therapy is more effective at eliciting a strong immune response.

To determine whether the strong IL12→IL27 immune gene therapy also induces a strong antitumor immune memory, the cured mice from both treatment groups, IL12→IL12 and IL12→IL27, were challenged with the tumor cells three months after the tumor disappearance. Interestingly, no tumor incidence was detected from the IL12→IL27-cured mice, suggesting the presence of a strong immune response. Tumor incidence was found in IL12→IL12-cured mice but these tumors were slowly regressed three weeks after the challenge, suggesting that the IL12→IL12 gene therapy also induced relatively weak anti-tumor immune response (FIG. 10E). The same challenge study was also performed in the mice 6 months after eradicating the aggressive 4T1 tumors by IL12→IL27 gene therapy. Tumors developed in 2 out of 5 challenged mice, but those two-developed tumors disappeared after 3 weeks, illustrating that this treatment yielded a long duration of anti-tumor memory against highly malignant 4T1 tumor cells. Since IL12→IL12 could not eradicate the aggressive 4T1 tumors, this challenge study could not be performed in IL12→IL12 treatment group.

Sequential gene therapy with IL12 followed by IL27 (both 10 μg DNA) was repeated in tumor-inoculated mice that were depleted of CD8 T, CD4 T, or NK cells using antibodies. The antibodies were given as 50 μg every three days, beginning three days prior to the administration of IL12 and ending two days prior to the administration of IL27. The effects on tumor size are shown in FIG. 11. As shown in FIG. 11, the administration of either antibody to CD8 and NK partially abrogated the tumor inhibition. These data support the conclusion above that a long term anti-tumor immune memory developed from the sequential gene therapy since depletion of CD8 T cells abrogated the systemic sequential IL12 and IL27 tumor eradication. The data indicate that sequential gene therapy induced tumor-specific T cells that eradicated the tumor and maintained the anti-tumor immune memory. This is a dramatic advancement over previous results that show IL12 gene therapy alone can only activate NK cells to inhibit tumor growth (4).

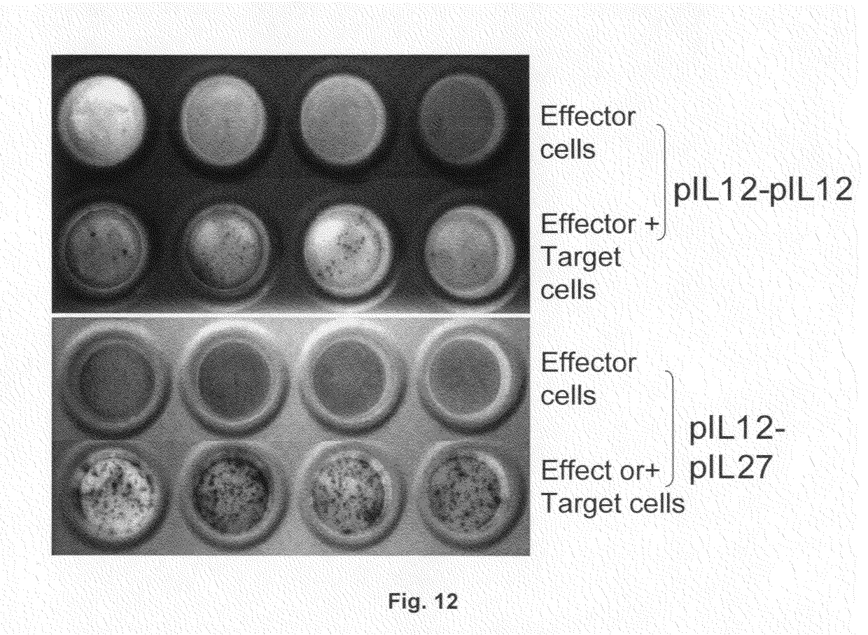

To investigate whether IL12→IL27-cured mice produce an acute induction of activated CD8 T cells to immediately remove the challenged tumor cells while IL12→IL12-cured mice may have only a slow response, IFN-γ-secreting CD8 T cells were identified from these two groups of mice following innoculation with tumor cells. To avoid the detection of the IFN-γ positive CD4 and NK cells, neutralization antibodies were administered two days prior to euthanizing mice. A much higher number of acute tumor-specific IFN-γ positive CD8 T cells were detected from IL12-IL27-cured mice than from IL12→IL12-cured mice (FIG. 12). The presence of a larger number of IFN-γ positive CD8 T cells was a good indicator for the acute induction of tumor specific CD8 T cells by the IL12→IL27 gene therapy.

### EXAMPLE 8

### Sequential IL12 and IL27 Gene Therapy is Tumor Specific

To determine whether the sequential IL12 and IL27 gene therapy would be effective in other types of tumors, two other tumor models, melanoma B16F10 and squamous cell carcinoma SCCVII, were tested. C3H mice were used as host for SCCVII tumors, and C57BL/6 mice were host for B16F10 tumors. The sequential administration of IL12→IL27 protocol was as reported in Example 2. Neither the melanoma nor the squamous cell carcinoma were eradicated using this sequential gene therapy. This indicates that the effectiveness of the sequential gene therapy is tumor specific (data not shown).

### EXAMPLE 9

### Expression of IL27 Receptor

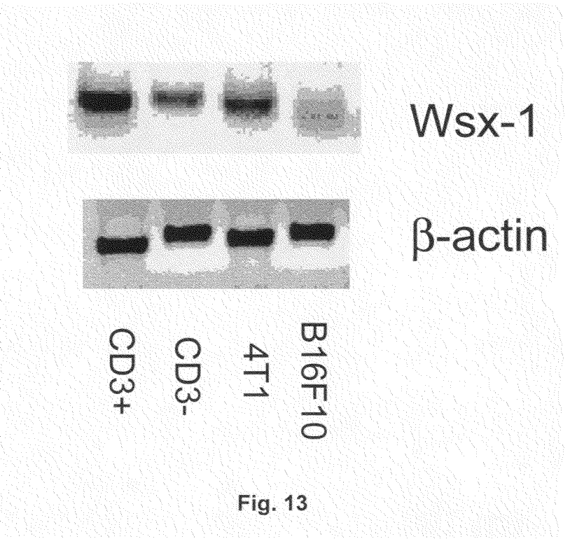

IL27 signaling typically requires the WSX-1 receptor. Thus, the expression WSX-1 in T cells and tumor cells was analyzed. Expression of the IL27 receptor was tested in CD3+ (activated lymphocyte cells with anti-CD3 antibody), CD3− (inactivated lymphocyte cells with anti-CD3 antibody), 4T1 (adenocarcinoma), and B16F10 (melanoma) cells by assaying for the WSX-1 protein. The IL27 receptor was expressed in the CD3+ cells as expected (FIG. 13). However, the IL27 receptor was expressed in the 4T1 tumor cells, but was not present or was present at low levels in the melanoma and SCCVII tumor cells (FIG. 13). Thus, the presence of WSX-1 correlated with tumor responsiveness to sequential gene therapy of IL12 followed by IL27.

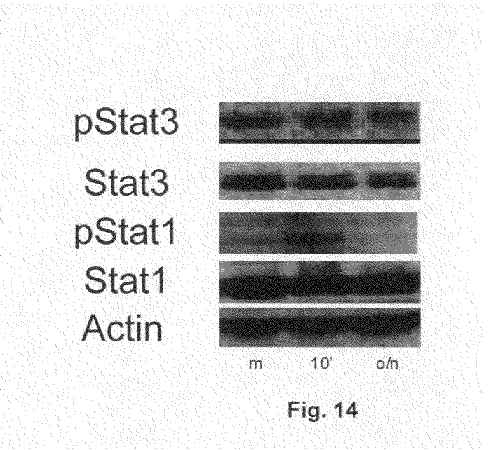

To further elucidate the effects of IL27, experiments were conducted to analyze the IL27 receptor signaling. The effect of IL27 on the expression of pStat1 (phosphorylated Stat1), Stat1, pStat3 (phosphorylated Stat3), and Stat 3 was analyzed in 4T1 tumor cells using Western Blot analysis. FIG. 14 illustrates the results of this analysis in cells grown in medium alone (left sample), in cells grown for 10 min in medium with IL27 (middle sample), and in cells grown overnight in medium with IL27 (right sample). IL27 was found to induce Stat1, but not Stat3 (FIG. 14). Increased activation of Stat1 has been shown to enhance tumor death by inducing multiple death gene expression (13).

To further evaluate the effects of IL27, experiments were conducted to analyze whether WSX-1 was activated by IL27. To accomplish this, the effects of IL27 on the expression of pStat1 (phosphorylated Stat1), Stat1, pStat3 (phosphorylated Stat3), and Stat 3 were analyzed in 4T1 tumor cells using Western Blot analysis as described in Example 1. FIG. 14 illustrates the results of this analysis in cells grown in medium alone (left sample), in cells grown for 10 min in medium with IL27 (middle sample), and in cells grown overnight in medium with IL27 (right sample). IL27 was found to induce pStat1, but not pStat3 (FIG. 14). An increased activation of pStat1 has been shown to enhance tumor death by inducing multiple death gene expression. (13)

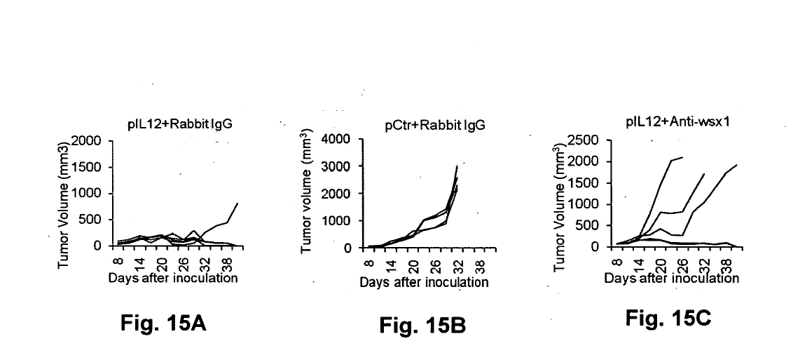

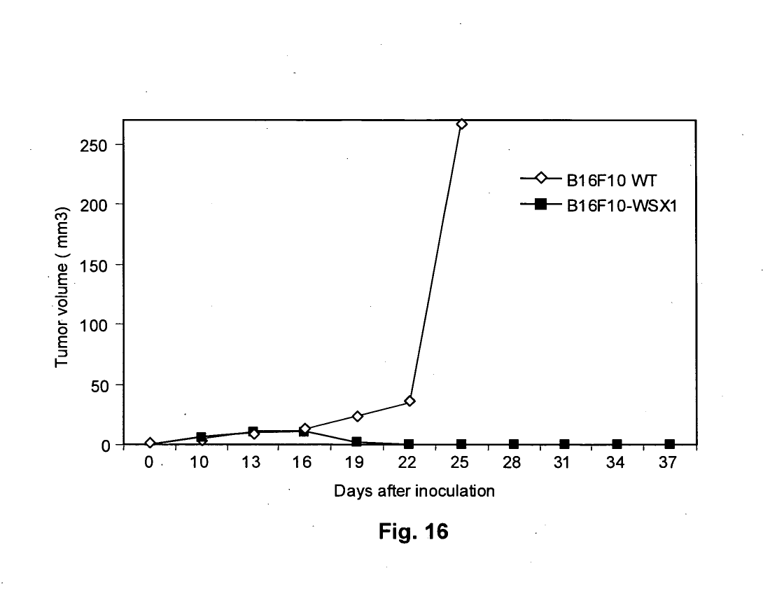

Our preliminary data indicated that WSX-1 is required for anti-tumor responsiveness to IL12 gene therapy which induces IL27 (FIG. 3F). Blocking the IL27 receptor wsx-1 impaired IL12-mediated tumor eradication. DNA encoding IL12 (pIL12) and control DNA (pCtrl) were administered into CT26 tumor bearing mice when tumors reached 5-7 mm in diameter (n=5) via intramuscular electroporation. A second administration was performed 10 days later. Rabbit IgG and human IgG were purchased from Sigma (St. Louis, Mo.). Anti-wsx-1, polyclonal IgG was isolated from wsx-1 immunized rabbits. Fifty μg IgG was administered into each mouse once every 3 days, from day 8 to day 32. Intramuscular administrations of IL12 vector by electroporation completely eradicated the established CT26 tumors from 80% of treated mice (FIG. 15A) but injection of the control DNA neither inhibited the aggressive tumor growth nor eradicated tumors (FIG. 15B). To test the effect of IL27 in response to IL12 gene therapy, polyclonal antibodies against IL27 were used to deplete WSX-1 from IL12 treated mice. Administration of WSX-1 antibody reduced the tumor regression to only 40% by (FIG. 15C). Moreover, tumor growth was more aggressive with the administration of WSX-1 antibody than administration of the control antibody in the IL12-treated mice (FIG. 15C). Together, these data collectively illustrate the dependence of IL27 by the IL12-mediated inhibition of tumor growth.

As shown above, the presence of the IL27 receptor is a predictor of which tumors will be responsive to the sequential gene therapy of IL12 and IL27. In a clinical setting, tumor samples can be analyzed for the IL27 receptor (e.g., analyze for WSX-1) to determine whether this sequential gene therapy might useful for treating residual malignancy. This analysis can either be by antibody binding or by functional production of Stat1.

### EXAMPLE 10

### WSX-1 is a Molecular Marker Useful for Selection of Tumors Responsive to IL12→IL27 Sequential Gene Therapy

A primary concern for IL12→IL27 therapy is its overall effectiveness. To be effective, tumors should express a cognate receptor capable of producing responsiveness to IL12→IL27 therapy. Here it was evaluated whether expression of the IL27 receptor WSX-1 might contribute to effectiveness of IL12→IL27 treatment.

Experiments were conducted to test the effects of IL12→IL27 administration in the presence or absence of WSX-1. Mouse xenograft models (C57B1/6 mice strain) were inoculated with 2×10⁵ wild type B16F10 melanoma cells (B16F10 WT; tumor cells without WSX-1) or 2×10⁵ B16F10 cells that stably express WSX-1 (B16F10-WSX-1). On day 10 after tumor inoculation, the mice were treated with IL12 plasmid DNA followed sequentially by IL27 10 days later. As shown in FIG. 16, the presence of WSX-1 correlated with the effectiveness of IL12→IL27 gene therapy (FIG. 16).

Next, the in vivo specificity of the IL12→IL27 gene therapy was evaluated using a panel of human tumor cell lines. Induction of Stat1 was monitored as an in vivo marker in these cell lines for responsiveness to IL27 treatment. Each cell line (5×10⁵ cells) were treated with IL27 (4 μg) for 10 minutes (10′) and overnight (o/n) or left untreated (m). In FIGS. 17A-17D, a western blot was probed with antibodies against pStat-1 tyr701, Stat-1, and actin, as previously described (16) and above in Example 1. As shown in FIGS. 17A-17D the breast cancer cell lines SKBR3 (FIG. 17A), MDA231 (FIG. 17B), and MDA453 (FIG. 17C) and the Jurkat T-cell lymphoma cell line (FIG. 17D) all increased the level of phosphorylated Stat1 (pStat1) at 10 min. In addition, as shown in FIGS. 17A-17D, the amount of Stat1 was increased in the overnight application of IL27. This result suggests that the ability of IL27 to phosphorylate Stat1 or to increase the amount of Stat1 may be used as a prognostic marker for tumors that possess a WSX-1 receptor and in which the IL12→IL27 gene therapy will be effective.

### REFERENCES

1. Wolff J A, Ludtke J J, Acsadi G. et al. Long-term persistence of plasmid DNA and foreign gene expression in mouse muscle. Hum Mol Genet. 1992; 1:363-9.
2. Mir L M. Bureau M F, Gehl J, et al. High-efficiency gene transfer into skeletal muscle mediated by electric pulses. Proc Natl Acad Sci USA. 1999; 96:4262-7.
3, Hanna E, Zhang X, Woodlis J, et al. Intramuscular electroporation delivery of IL-12 gene for treatment of squamous cell carcinoma located at a distant site. Ca Gene Ther. 2001; 8:1-7.
4. Li S, Zhang L, Torrero M., et al. Administration route- and immune cell activation-dependent tumor eradication by IL12 electrotransfer. Mol Ther. 2005; 112:942-9.
5. Li S. IL-12-based therapy of malignancies. Drugs Today (Barc). 2001; 37:629-37.
6. Tan J, Newton C A, Djeu J Y, et al. Injection of complementary DNA encoding interleukin-12 inhibits tumor establishment at a distant site in a murine renal carcinoma model. Cancer Res. 1996; 56:3399-403.
7. Rosenzweig S D, Schwartz O M, Brown M R, et al. Characterization of a dipeptide motif regulating IFN-gamma receptor 2 plasma membrane accumulation and IFN-gamma responsiveness. J Immunol. 2004; 173:3991-9.
8. Li S, Zhang X, Xia X. Regression of tumor growth and induction of long-term antitumor memory by interleukin 12 electro-gene therapy. J Natl Cancer Inst. 2002; 94:762-8.
9. Salcedo R, Stauffer J K, Lincoln E, et al. IL-27 mediates complete regression of orthotopic primary and metastatic murine neuroblastoma tumors: Role for CD8′ T cells. J Immunol. 2004; 173:7170-82.
10. Morishima N, Owaki T, Asakawa M, et al. Augmentation of effector CD8⁺ t cell generation with enhanced granzyme B expression by IL27. J Immunol. 2005; 175:1686-93.
11. Pulaski B A and Ostrand-Rosenberg S. Reduction of established spontaneous mammary carcinoma metastases following immunotherapy with major histocompatibility complex class 11 and B7.1 cell-based tumor vaccines. Cancer Res. 1998; 58:1486-93.
12. Pulaski B A, Terman D S, Khan S, et al. Cooperativity of Staphyococcal aureus enterotoxin B superantigen, major histocompatibility complex class II, and CD80 for immunotherapy of advanced spontaneous metastases in a clinically relevant postoperative mouse breast cancer model. Cancer Res. 2000; 60:2710-5.
13. Torrero M N, Xia X, Henk W, et al. Stat1 deficiency in the host enhances interleukin-12-mediated tumor regression. Cancer Res. 2006; 66:4461-7.
14. Li S, Zhang X, Xia X, et al. Intramuscular electroporation delivery of IFN-alpha gene therapy for inhibition of tumor growth located at a distant site. Gene Ther. 2001; 8:400-07.
15. Liu L, Chahroudi A, Silvestri G, et al. Visualization and quantification of T cell-mediated cytotoxicity using cell-permeable fluorogenic caspase substrates. Nat Med. 2002; 8:185-9,
16. Li S, Xia X, Zhang X, et al. Regression of tumors by IFN-alpha electroporation gene therapy and analysis of the responsible genes by cDNA array. Gene Ther, 2002; 9:390-7,
17. Zagozdzon R, Golab J, Stoklosa T, et al. Effective chemo-immunotherapy of I¹²¹0 leukemia in vivo using interleukin-12 combined with doxorubicin but not with cyclophosphamide, paclitaxel or cisplatin. Int J Cancer. 1998; 77:720-7.
18 Torrero M N, Henk W O, Li S. Regression of high-grade malignancy in mice by bleomycin and interleukin-12 electrochemogenetherapy. Clin. Cancer Res. 2006; 12:257-63.
19. Pflanz S, Hibbert L, Mattson J, et al. WSX-1 and glycoprotein 130 constitute a signal-transducing receptor for 1 L-27. J. Immuno, 2004; 172:225-331
20. Liu, J., X. Xia, M. Torrero, R. Barrett, E. J. Shillitoe, and S. Li. the mechanism of exogenous B7.1-enhanced IL-12 medicated complete regression of tumors by a single electroporation delivery. Int. J. Cancer 2006; 119:2113-2118.

The complete disclosures of all references cited in this specification are hereby incorporated by reference. In the event of an otherwise irreconcilable conflict, however, the present specification shall control.

We claim:

1. A method to inhibit one or more tumors or metastases in a mammal, said method comprising the sequential steps of: (a) administering to the mammal an effective amount of IL12; (b) waiting for a time longer than about 24 hours; and (c) administering to the mammal an effective amount of IL27, wherein the tumors or metastases decrease more than would be the case for an otherwise identical method that lacks step (c).

2. A method as in claim 1, wherein the tumors or metastases express a receptor for IL27.

3. A method as in claim 1, wherein step (b) comprises waiting longer than about five days.

4. A method as in claim 1, wherein step (b) comprises waiting longer than about ten days.

5. A method as in claim 1, wherein the IL12 and IL27 are administered by introducing into the mammal one or more exogenous nucleic acid constructs encoding either IL12 or IL27, in a manner permitting expression of IL12 or IL27.

6. A method as in claim 5, wherein the nucleic acid construct is introduced into a tumor, into the skin, or into a muscle of the mammal.

7. A method as in claim 6, wherein the nucleic acid construct is introduced into a muscle.

8. A method to inhibit the growth of metastatic tumors in a mammal with a malignant tumors said method comprising the sequential steps of, (a) administering to the mammal an effective amount of IL12; (b) waiting for a time longer than about 24 hours; and (c) administering to the same mammal an effective amount of IL27; wherein the number of metastatic tumors decreases more than would be the case for an otherwise identical method that lacks step (c).

9. A method as in claim 8, wherein the malignant tumors express a receptor for IL27.

10. A method as in claim 8, wherein step (b) comprises waiting longer than about five days.

11. A method as in claim 8, wherein step (b) comprises waiting longer than about ten days.

12. A method as in claim 8, wherein the IL12 and IL27 are administered by introducing into the mammal one or more exogenous nucleic acid constructs encoding either IL12 or IL27, in a manner permitting expression of IL12 or IL27.

13. A method of claim 12, wherein the nucleic acid construct is introduced into a tumor, into the skin, or into a muscle of the mammal.

14. A method of claim 13, wherein the nucleic acid is introduced into a muscle.

15. A method to diagnose the likely responsiveness of a tumor to the sequential administration of IL12 followed by the administration of IL27, said method comprising analyzing cells from the tumor for the presence of a WSX-1 receptor, wherein the presence of a WSX-1 receptor indicates that the tumor will likely respond to the sequential administration of IL12 followed by the administration of IL27.

16. A method as in claim 15, wherein said analyzing step comprises administering IL27 to the tumor cells, and assaying any change in the phosphorylation state of Stat1, wherein an increase in phosphorylated Stat1 indicates that the tumor will likely respond to the sequential administration of IL12 followed by administration of IL27.

17. A method as in claim 15, wherein said analyzing step comprises administering IL27 to the tumor cells, and assaying any change in the Stat1 expression % wherein an increase in Stat1 expression indicates that the tumor will likely respond to the sequential administration of IL12 followed by administration of IL27.

18. A method to diagnose the likely responsiveness of a tumor to the administration of IL12, said method comprising analyzing cells from the tumor for the presence of a WSX-1 receptor; wherein the presence of a WSX-1 receptor indicates that the tumor will likely respond to the administration of IL12.

19. A method as in claim 18, wherein said analyzing step comprises administering IL27 to the tumor cells, and assaying any change in the phosphorylation state of Stat1, wherein an increase in phosphorylated Stat1 indicates that the tumor will likely respond to the sequential administration of IL12 followed by administration of IL27.

20. A method as in claim 18, wherein said analyzing step comprises administering IL27 to the tumor cells, and assaying any change in the Stat1 expression, wherein an increase in Stat1 expression indicates that the tumor will likely respond to the sequential administration of IL12 followed by administration of IL27.


SEQUENCE LISTING (12 sequences)

SEQ ID NO: 1 - DNA, 20 bp, Artificial Sequence
  Synthetic Primer.
atgaacgctacacactgcat

SEQ ID NO: 2 - DNA, 18 bp, Artificial Sequence
  Synthetic Primer
tcagcagcgactcctttc

SEQ ID NO: 3 - DNA, 20 bp, Artificial Sequence
  Synthetic Primer
tcatgaacaacaggtggatc

SEQ ID NO: 4 - DNA, 20 bp, Artificial Sequence
  Synthetic Primer
caggtcttcagttttggaag

SEQ ID NO: 5 - DNA, 20 bp, Artificial Sequence
  Synthetic Primer
atgctggattgcagagcagt

SEQ ID NO: 6 - DNA, 20 bp, Artificial Sequence
  Synthetic Primer
tgggcatccttaagctgttg

SEQ ID NO: 7 - DNA, 20 bp, Artificial Sequence
  Synthetic Primer
caagaagaggtcccgtgctg

SEQ ID NO: 8 - DNA, 20 bp, Artificial Sequence
  Synthetic Primer
ttgagcccagtccaccacat

SEQ ID NO: 9 - DNA, 21 bp, Artificial Sequence
  Synthetic Primer
gtgggccgccctaggcaccag

SEQ ID NO: 10 - DNA, 23 bp, Artificial Sequence
  Synthetic Primer
tctttgatgtcacgcacgatttc

SEQ ID NO: 11 - DNA, 25 bp, Artificial Sequence
  Synthetic Primer
cggatccatgaaccggctccgggtt

SEQ ID NO: 12 - DNA, 21 bp, Artificial Sequence
  Synthetic Primer
tcagactagaaggcccagctc